(19) United States
(12) Patent Application Publication
(10) Pub. No.: US 2017/0165196 A1
(43) Pub. Date: Jun. 15, 2017

(54) Cosmetic Cream Formulation

(71) Applicants: Diana Nattrass, Bethpage, NY (US); Bessie Brewster, Jamaica, NY (US); Ralph Brewster, Jamaica, NY (US)

(72) Inventors: Diana Nattrass, Bethpage, NY (US); Bessie Brewster, Jamaica, NY (US); Ralph Brewster, Jamaica, NY (US)

(21) Appl. No.: 15/339,896

(22) Filed: Oct. 31, 2016

Related U.S. Application Data
(60) Provisional application No. 62/248,643, filed on Oct. 30, 2015.

(51) Int. Cl. A61K 8/98; A61Q 19/00; A61Q 19/08; A61K 8/97

(52) U.S. Cl. CPC A61K 8/988; A61K 8/97; A61Q 19/007; A61Q 19/08

(57) ABSTRACT

A cosmetic cream formulation for cleaning, restoring, replenishing, and moisturizing a user's skin is provided. The cosmetic cream formulation comprises a cosmetic cream having one part pumpkin and one part honey. The cosmetic cream revitalizes the user's skin to a more healthy and youthful feeling, tone and appearance.

### CLAIM OF PRIORITY

This patent application claims priority under 35 USC 119 (e) (1) from U.S. Provisional Patent Application Ser. No. 62/248,587 filed Oct. 30, 2015, of common inventorship herewith entitled, “Punkin Pretty,” which is incorporated herein by reference as though the same were set forth in its entirety.

### FIELD OF THE INVENTION

The present invention pertains to the field of organic cosmetics, and more specifically to the field of all natural facial masks.

### BACKGROUND OF THE INVENTION

The skin is a human being's single biggest organ and the organ most exposed to the outside world. Throughout one's life, an individual's skin is continually renewed cell by cell, but the effects of sunlight and ultraviolet radiation, gravity, and habits of consumption do leave their marks. The facial skin becomes lined and wrinkled around one's mouth and eyes, liver spots and other chronic discolorations appear, and precancerous or cancerous conditions of the skin may develop. Developing treatments for skin go far back in history.

No one knows when or where humanity first discovered the tactile, sensual cleansing, and aesthetic benefits of a hot mud facial treatment, but best bets center on groups of hunter gatherers who dwelt in or passed through regions of geothermal activity such as hot mineral springs, mud pots, and geyser basins. Age old thermal, mineral, and mud treatments have been passed down from such places and peoples around the globe. Until comparatively recent times, the treatments were considered exotic and limited to those persons who had initiative and wealth to journey to exclusive spas and hot springs.

Today, any woman or man who desires a more youthful, healthier appearance can undergo a wide variety of beauty enhancing treatments from a hot mud facial to Botox injections to plastic surgery. The search for healthier, more youthful skin need not involve such drastic measures. A simple facial in a jar can confer great benefit to the skin from a first application of approximately thirty minutes.

The prior art has put forth several designs for all natural facial masks. Among these are:

US Patent Publication 2013/0058885 to Johnny Burt, Kathy Burt and Tyler Goodwin describes embodiments that relate to novel personal care compositions for cleaning and moisturizing the skin or hair comprising an extract of squash and pumpkin, a preservative and a cosmetic base. Other embodiments of the present invention relate to methods of making and methods of using the personal care compositions.

U.S. Pat. No. 8,110,207 to Juan Cebrian Puche, Angel Messeguer Peypoch, Antonio Ferrer Montiel, Nuria Alminana Domenech and Cristina Carreno describes cosmetic or pharmaceutical compositions containing compounds for treating, caring for and cleaning skin, hair and nails, preferably for attenuating the degree of pigmentation of the skin and the hair or for treating spots on nails or for photo protection of the skin, hair and nails.

US Patent Publication 2010/0233128 to Oksana Panasenko describes a skincare product containing one or more dairy products, and one or more ingredients such as a natural oil, an herb, a fruit, a vegetable, a grain, honey, sea salt, egg yolk and mixtures thereof, for application to human skin in order to improve the wellbeing of said skin. The skincare product is applied to the skin to cleanse, beautify, promote attractiveness, and alter the feel or appearance of the skin. The skincare product is applied to the skin by rubbing, pouring, sprinkling or spraying the skincare product on to the skin.

None of these prior art references describe the present invention.

### SUMMARY OF THE INVENTION

It is an object of the present invention to provide a cosmetic mixture, particularly formulated as a facial mask, to clean, restore, replenish and moisturize a user's skin.

The present invention is a cosmetic cream formulation for cleaning, restoring, replenishing, and moisturizing a user's skin. The cosmetic cream formulation comprises a cosmetic cream having one part pumpkin and one part honey. The cosmetic cream revitalizes the user's skin to a more healthy and youthful feeling, tone and appearance.

In addition, the present invention includes a method for cleaning, restoring, replenishing, and moisturizing a user's skin. The method comprises providing a cosmetic cream having one part pumpkin and one part honey, applying the cosmetic cream to the user's skin, and revitalizing the user's skin to a more healthy and youthful feeling, tone and appearance.

The present invention further includes a cosmetic cream formulation for cleaning, restoring, replenishing, and moisturizing a user's skin. The cosmetic cream formulation comprises a cosmetic cream having one part pumpkin and one part honey and a portion of cinnamon spice blended into the cosmetic cream. Upon applying the cosmetic cream to the user's skin for approximately thirty minutes, the cosmetic cream revitalizes the user's skin to a more healthy and youthful feeling, tone and appearance.

### BRIEF DESCRIPTION OF THE DRAWINGS

FIG. 1 is a front perspective view illustrating a cosmetic cream of a cosmetic cream formulation, constructed in accordance with the present invention, with the cosmetic cream stored inside a closed container with a logo name printed across the container.

FIG. 2 is a top down perspective view illustrating the cosmetic cream of the cosmetic cream formulation, constructed in accordance with the present invention, with the cosmetic cream comprised of a mixture of pumpkin, honey, and cinnamon spice in an opened container.

FIG. 3 is another front view perspective view illustrating the cosmetic cream of the cosmetic cream formulation, constructed in accordance with the present invention, with the cosmetic cream stored inside a container with its logo name printed across the container.

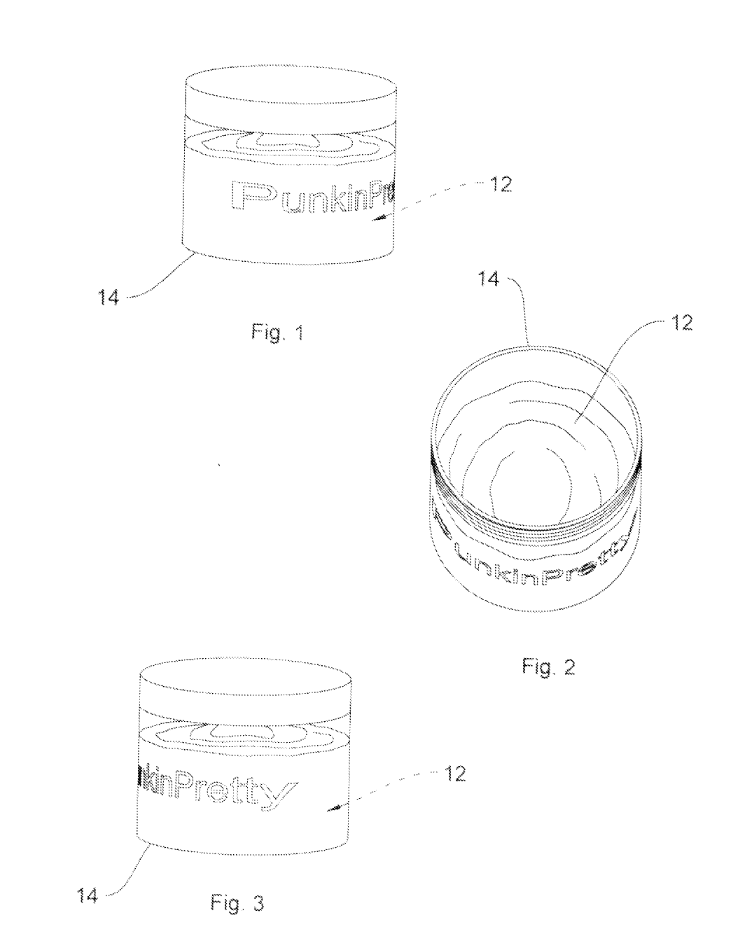

### DETAILED DESCRIPTION OF THE INVENTION

The present invention, hereinafter referred to as a Cosmetic Cream Formulation, indicated generally at 10, is a cosmetic mixture 12, particularly formulated as a facial mask, whose primary function is to clean, restore, replenish, and moisturize a user's skin. The Cosmetic Cream Formulation 10 revitalizes the user's skin to a more healthy and youthful feeling, tone and appearance with each application.

The Cosmetic Cream Formulation 10 comprises a specially formulated facial paste or cosmetic cream 12 blending pumpkin, honey, and cinnamon spice to promote a smooth, replenished, toned, and moisturized skin. The formula of the cosmetic cream 12 uses organic pumpkin and honey in a one to one, half and half ratio, with a portion of cinnamon spice blended in. The amount of cinnamon spice added to the combined pumpkin and honey cosmetic cream 12 is enough to impart a pleasing aroma to the formulation. The formula of the cosmetic cream 12 of the Cosmetic Cream Formulation 10 facial is preferably stored in wide mouthed jars or bottles 14 and available in several different volumes.

Application and use of the Cosmetic Cream Formulation 10 of the present invention is simple and straightforward. The user simply washes and dries their face, applies the Cosmetic Cream Formulation 10 paste or cream 12, leaves the facial mask in place for approximately thirty minutes, and then wipes it off with a damp washcloth. With each application, the cosmetic cream 12 of the Cosmetic Cream Formulation 10 cleans the skin of the face, restores the skin's essential oils and vitamins, refreshes the skin, and smooths out the wrinkles, lines, and blemishes caused by age and sun. Natural and organic, the ingredients used in the cosmetic cream 12 of the Cosmetic Cream Formulation 10 restore natural, youthful elasticity of the skin, eliminate dark spots and bags beneath the eyes, and provide a smooth toning effect for a healthy glowing appearance. Providing a deep cleaning of the pores of the skin, the cosmetic cream 12 of the Cosmetic Cream Formulation 10 helps to prevent the return of blemishes such as blackheads and pimples.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A cosmetic cream formulation for cleaning, restoring, replenishing, and moisturizing a user's skin, the cosmetic cream formulation comprising:
a cosmetic cream having one part pumpkin and one part honey;
wherein the cosmetic cream revitalizes the user's skin to a more healthy and youthful feeling, tone and appearance.

2. The cosmetic cream formulation of claim 1 wherein the cosmetic cream further comprises:
a portion of cinnamon spice blended into the cosmetic cream.

3. The cosmetic cream formulation of claim 1 wherein the cosmetic cream is a facial mask.

4. The cosmetic cream formulation of claim 1 wherein the cosmetic cream is stored in jars or bottles.

5. The cosmetic cream formulation of claim 4 wherein the jars or bottles are wide mouthed jars or bottles.

6. The cosmetic cream formulation of claim 4 wherein the jars or bottles are available in several different volumes.

7. The cosmetic cream formulation of claim 1 wherein the cosmetic cream is applied to the user's skin for approximately thirty minutes.

8. The cosmetic cream formulation of claim 1 wherein the cosmetic cream restores the user's skin's essential oils and vitamins.

9. The cosmetic cream formulation of claim 1 wherein the cosmetic cream refreshes the user's skin, and smooths out the wrinkles, lines, and blemishes.

10. The cosmetic cream formulation of claim 1 wherein the cosmetic cream is natural and organic.

11. A method for cleaning, restoring, replenishing, and moisturizing a user's skin, the method comprising:
providing a cosmetic cream having one part pumpkin and one part honey;
applying the cosmetic cream to the user's skin; and
revitalizing the user's skin to a more healthy and youthful feeling, tone and appearance.

12. A cosmetic cream formulation for cleaning, restoring, replenishing, and moisturizing a user's skin, the cosmetic cream formulation comprising:
a cosmetic cream having one part pumpkin and one part honey;
a portion of cinnamon spice blended into the cosmetic cream;
wherein upon applying the cosmetic cream to the user's skin for approximately thirty minutes, the cosmetic cream revitalizes the user's skin to a more healthy and youthful feeling, tone and appearance.

13. The cosmetic cream formulation of claim 12 wherein the cosmetic cream is a facial mask.

14. The cosmetic cream formulation of claim 12 wherein the cosmetic cream is stored in jars or bottles.

15. The cosmetic cream formulation of claim 14 wherein the jars or bottles are wide mouthed jars or bottles.

16. The cosmetic cream formulation of claim 14 wherein the jars or bottles are available in several different volumes.

17. The cosmetic cream formulation of claim 12 wherein the cosmetic cream restores the user's skin's essential oils and vitamins.

18. The cosmetic cream formulation of claim 12 wherein the cosmetic cream refreshes the user's skin, and smooths out the wrinkles, lines, and blemishes.

19. The cosmetic cream formulation of claim 12 wherein the cosmetic cream is natural and organic.
